(19) United States
(12) Patent Application Publication
(10) Pub. No.: US 2015/0272848 A1
(43) Pub. Date: Oct. 1, 2015

(54) Topical skin care formulations

(71) Applicant: Louise Holyfield, Dallas, TX (US)

(72) Inventor: Louise Holyfield, Dallas, TX (US)

(21) Appl. No.: 14/544,089

(22) Filed: Nov. 24, 2014

Related U.S. Application Data
(63) Continuation-in-part of application No. 13/999,849, filed on Mar. 27, 2014, now Pat. No. 9,060,946.

(51) Int. Cl. A61K 8/49; A61Q 19/08; A61K 8/29; A61K 8/06; A61Q 17/04; A61K 8/37

(52) U.S. Cl. CPC A61K 8/4966; A61Q 17/04; A61K 8/37; A61K 8/29; A61K 8/06; A61Q 19/08; A61K 2800/42; A61K 2800/524

(57) ABSTRACT

Stable whiteness is provided by adding stabilizers to a topical anti-aging, anti-oxidant sunscreen or skin care formulation containing Tinosorb S and other optional ingredients, including CoQ10. The formulation will not stain clothing or other fabrics.

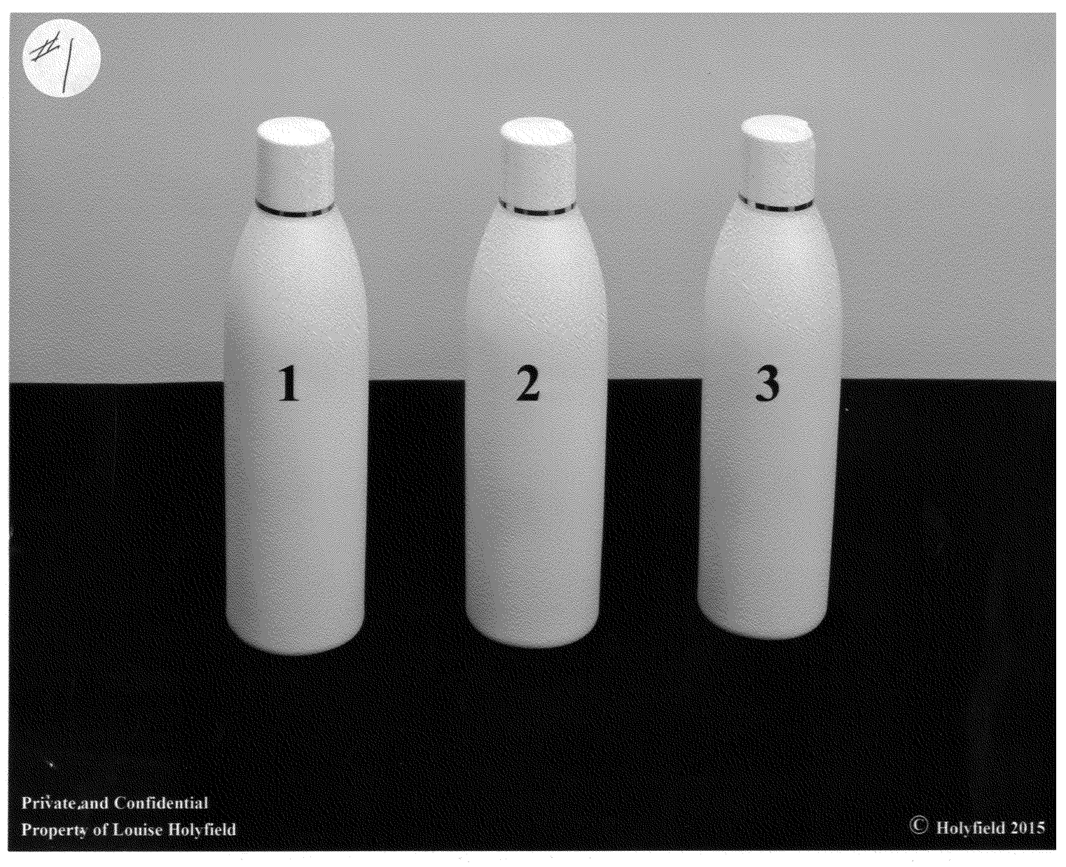

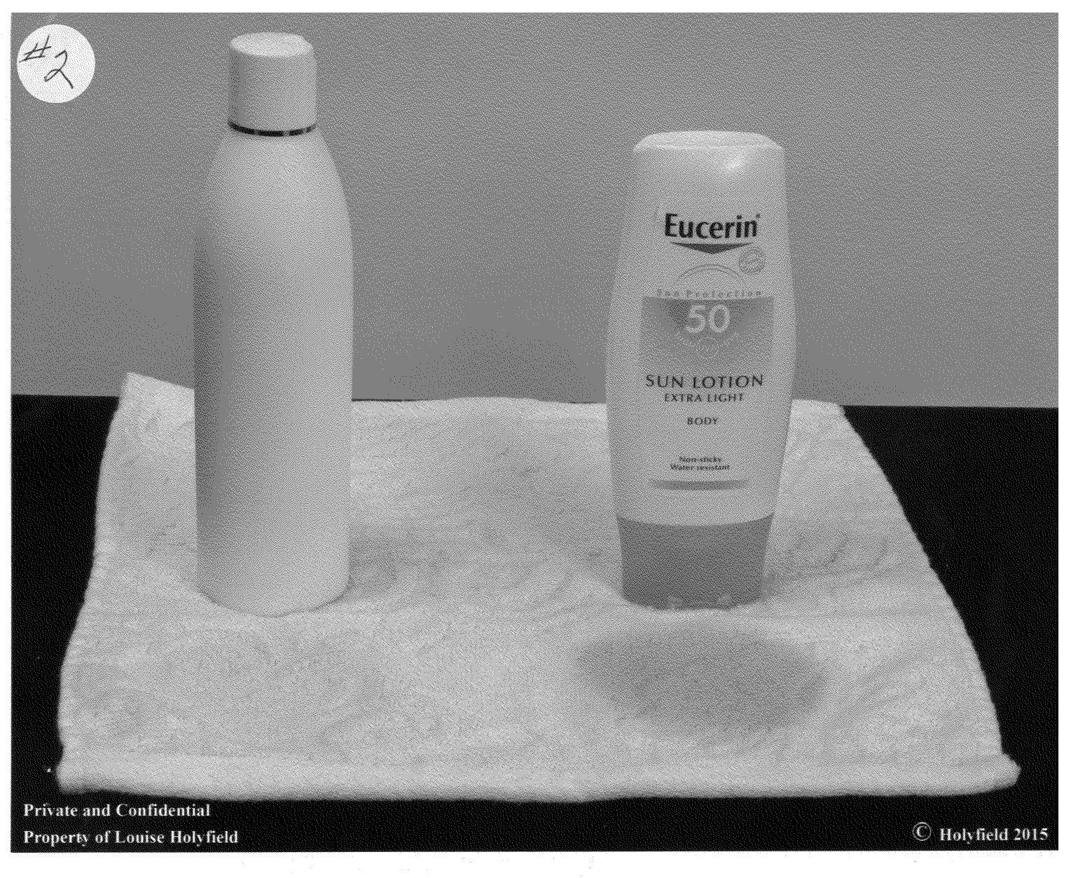

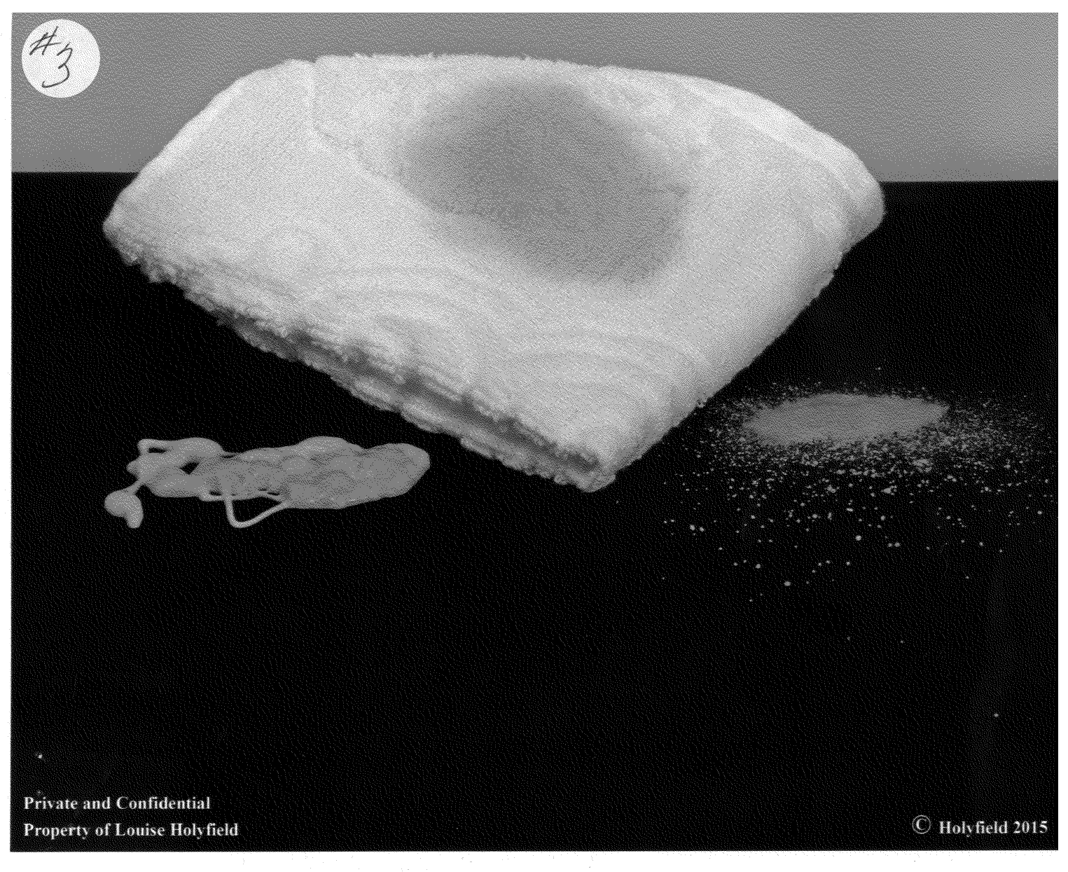

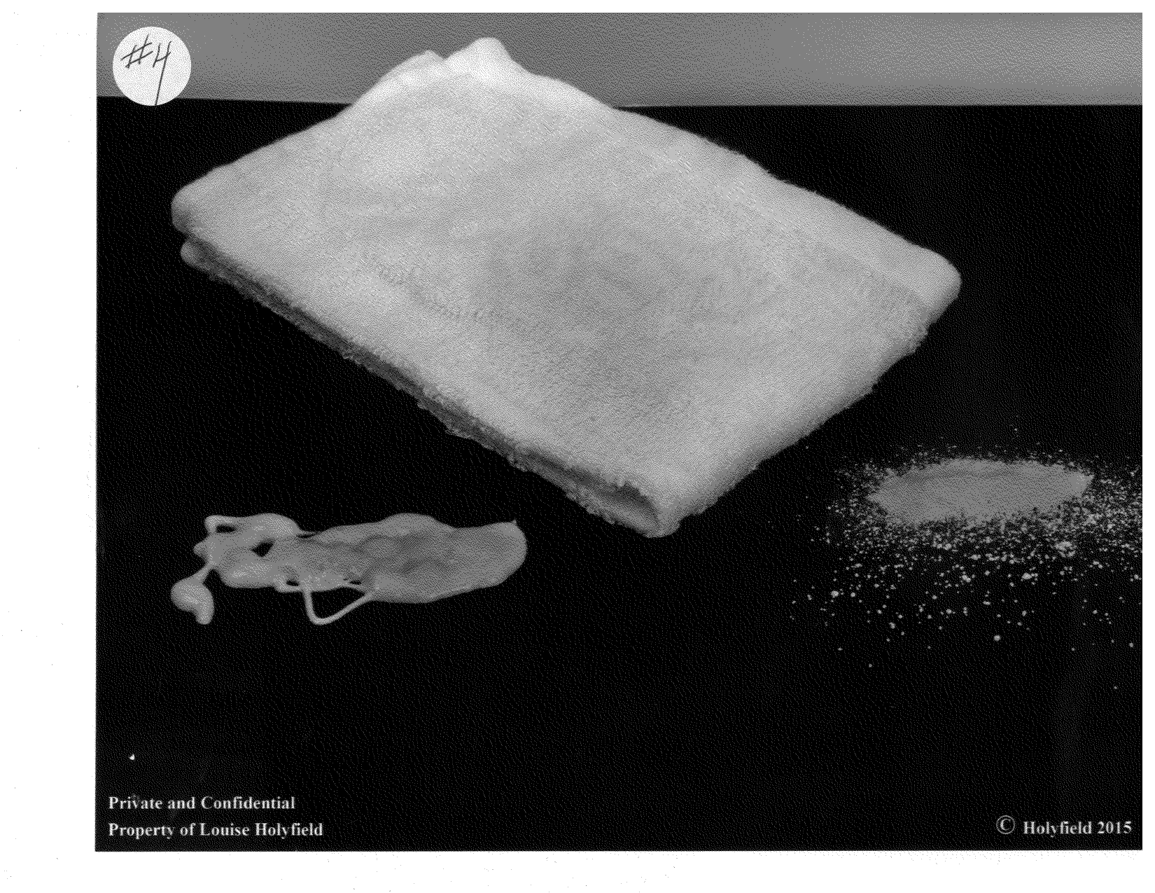

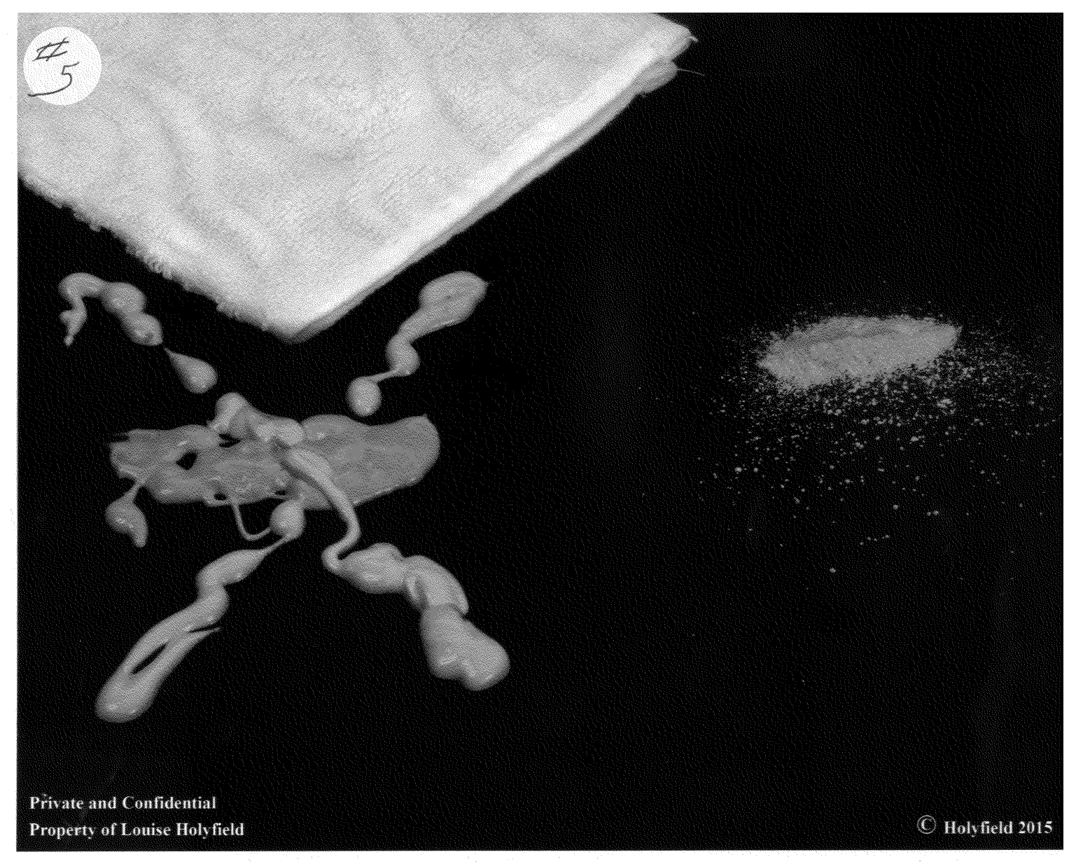

This application is a continuation-in-part of application Ser. No. 13/999,849 filed Mar. 27, 2014, which is entitled in part to the benefit of the filing date of U.S. Pat. No. 8,314,154 filed Sep. 22, 2008, incorporated herein by reference.

### FIELD OF INVENTION

This invention relates to chemical compositions, and more particularly to topical anti-aging, anti-oxidant, sunscreen, and skin care products and methods of use.

### BACKGROUND OF INVENTION

Research has confirmed the benefits of proper skin care through the use of products that contain anti-aging ingredients, primarily anti-oxidants and sunscreens.

Consumers, both men and women regardless of age are particularly interested in products that enhance the appearance of their skin while protecting it from free radicals, stress oxidation, and the sun.

Anti-oxidants and sunscreens are the skin's first defense in fighting free radicals that are in the form of ionic particles that persist in the atmosphere. Free radicals have the ability to permeate the skin and lungs thereby damaging skin cells/DNA.

As world population grows there is every indication that the demand for sunscreens and skin care products that contain sunscreens will grow exponentially.

The first signs of aging are reflected in the number of wrinkles a person has and the visual sun-damage their skin projects. An extreme example would be to compare the skin appearance of an average Certified Public Accountant to that of a professional surfer. Sun damage to the skin is irreversible.

We live in a society that admires youth and beauty and having a tan in many cultures is considered to be a sign of beauty. In the United States the highest percentage of individuals being diagnosed with melanoma (the deadliest form of skin cancer) are between the ages of 15 and 29. Statistics regarding the annual increase in cases of skin cancer in younger individuals does not paint a pretty picture for anyone who tans, over-tans, or uses tanning beds for the purpose of looking more attractive. Being tanned can come at a very high price, be it short term or long term.

Chemical UVA+UVB sunscreen/sunblock Bemotrizinol (Tinosorb S) is a broad spectrum sunscreen ingredient that is used in sunscreen and skin care products. Broad spectrum product labeling indicates that the product has sunscreen ingredients that block/filter both UVA and UVB rays.

The chemical Bemotrizinol (Tinosorb S) is yellow in color. When the chemical has been used in a sunscreen or skin care formulation the product has also been yellow in color. Degradation of the yellow color has continued during the shelf life of any product containing the chemical.

The use of Bemotrizinol (Tinosorb S) in a sunscreen or skin care formulation has caused permanent staining to any clothing that it contacted.

White is the commercially preferred color for any sunscreen or skin care formulation used on the face and body. White represents purity to the consumer.

To optimize the benefits of Bemortrizinol (Tinosorb S), in sunscreens and skin care formulations it is imperative that the product formulation be white in color and remain white in color during the shelf life of a product formulation.

### SUMMARY OF THE INVENTION

One aspect of the invention provides a white, color-stable topical anti-aging, anti-oxidant sunscreen or skin care formulation comprising 1) an effective amount of Bemotrizinol (Tinosorb S), or a derivative thereof, in an aqueous emulsion formulation that is white in color and remains white in color during the shelf life of the product, and 2) will not cause permanent staining to clothing or other fabrics during the shelf life of the formulation.

A second aspect of the invention provides a white, color stable topical anti-aging, anti-oxidant sunscreen or skin care formulation comprising 1) an effective amount of Bemotrizinol (Tinosorb S), or a derivative thereof, in an aqueous emulsion formulation that is white in color and remains white in color during the shelf life of the product, and 2) will not cause permanent staining to clothing or other fabrics during the shelf life of the formulation.

Due to unexpected results the third aspect of the invention provides a white, color stable topical anti-aging, anti-oxidant sunscreen or skin care formulation comprising of 1) an amount of Bemotrizinol (Tinosorb S), or a derivative thereof, and 2) an amount of CoQ10, or a derivative thereof, in an aqueous emulsion formulation that is white in color and that will remain white in color during the shelf life of the product formulation, and 3) formulation not cause permanent staining to clothing during the shelf life of the formulation.

Due to additional unexpected results the fourth aspect of the invention provides a white, color stable topical anti-aging, anti-oxidant sunscreen or skin care formulation comprising of 1) an amount of Bemotrizinol (Tinosorb S), or a derivative thereof, and 2) an amount of CoQ10, or a derivative thereof, and 3) an amount of Vanillin, or a derivative thereof, in an aqueous emulsion formulation that is white in color and that will remain white in color during the shelf life of the product formulation, and 4) formulation not cause permanent staining to clothing during the shelf life of the formulation.

In order to preserve whiteness when using Bemotrizinol (Tinosorb S), or a derivative thereof, additional ingredients are added to the above formulation. The preferred additional ingredients are octyl salicylate and octyl methoxycinnamate and titanium dioxide. Other such ingredients include:

p-aminobenzoic acid (PABA)

octyldimethylPABA

phenylbenzimidazolesulfonic acid

2-ethoxyethyl p-methoxycinnamate

Benzophenone

homomethyl salicylate

methyl anthranilate

2-cyano-3,3-diphenyl acrylic acid

triethanolamine salicylate

butylmethoxy dibenzoylmethane

4-methylbenzylidene camphor

disodium phenyl dibenzimidazole tetrasulfonate

### EXAMPLES

A. 10% by wt. Bemotrizinol (Tinosorb S)
   5% by wt. Octyl salicylate
   5% by wt. Octyl methoxycinnamate
   0.5% by wt. Titanium dioxide
   79.5% by wt. white emulsion, including water, oil and emulsifiers
B. 8% by wt. Bemotrizinol (Tinosorb S)
   5% by wt. Octyl salicylate
   5% by wt. Octyl methoxycinnamate
   0.5% by wt. Titanium dioxide
   81.5% by wt. white emulsion, including water, oil, and emulsifiers
C. 4% by wt. Bemotrizinol (Tinosorb S)
   3% by wt. Octyl salicylate
   3% by wt. Octyl methoxycinnamate
   0.5% by wt. Titanium dioxide
   89.5% by wt. white emulsion, including water, oil, and emulsifiers
D. 0.01% by wt. Bermotrizinol (Tinosorb S)
   2% by wt. Octyl salicylate
   3% by wt. Octyl methoxycinnamate
   0.5% by wt. Titanium dioxide
   94.49% by wt. white emulsion, including water, oil, and emulsifiers
E. 10% by wt. Bemotrizinol (Tinosorb S)
   3% by wt. CoQ10
   5% by wt. Octyl salicylate
   5% by wt. Octyl methoxycinnamate
   0.75% by wt. Titanium Dioxide
   76.25% by wt. white emulsion, including water, oil, and emulsifiers
F. 10% by wt. Bemotrizonal (Tinosorb S)
   4% by wt. Vanillin
   5% by wt. Octyl Salicylate
   5% by wt. Octyl methoxycinnamate
   0.75% by wt. Titanium Dioxide
   75.25% by wt. white emulsion, including water, oil, and emulsifiers
G. 10% by wt. Bemotrizonal (Tinosorb S)
   3% by wt. CoQ10
   4% by wt. Vanillin
   6% by wt. Octyl salicylate
   6% by wt. Octyl Methoxycinnamate
   0.75% by wt. Titanium Dioxide
   70.25 by wt. white emulsion, including water, oil, and emulsifiers.

Examples A-G of the above formulations ensure that each formulation is white in color and will remain white in color during the shelf life of the formulation and that formulation should be classified as an anti-aging, anti-oxidant, sunscreen formulations containing SPF.

1. A white, color-stable, topical anti-aging anti-oxidant skin care formulation comprising:
a white emulsion including water, oil, and at least one emulsifier;
an effective amount of bemotrizinol for sunscreen protection; and
effective amounts of octyl salicylate, octyl methoxycinnamate and titanium dioxide for preservation of whiteness; and
said formulation having stable whiteness for its entire shelf life, and having no ability to cause staining while in contact with clothing and other fabrics.

2. A formulation as in claim 1 wherein the amount of bemotrizinol is within the range of 0.01% to about 10% by wt.

3. A formulation as in claim 1 wherein the amount of octyl salicylate is within the range of 1.0% to 10% by wt.

4. A formulation as in claim 1 wherein the amount of octyl methoxycinnamate is within the range of 1.0% to 10% by wt.

5. A formulation as in claim 1 wherein the amount of titanium dioxide is within the range of 0.1% to 2.0% by wt.

6. A formulation as in claim 1 further including an effective amount of CoQ10 for anti-aging.

7. A formulation as in claim 1 further including an effective amount of vanillin as an antioxidant.

8. A formulation as in claim 1 wherein the ingredient percentages are:

| | |
|---|---|
| white emulsion | 79.5% by wt |
| bemotrizinol | 10% by wt |
| octyl salicylate | 5% by wt |
| octyl methoxycinnamate | 5% by wt |
| titanium dioxide | 0.5% by wt |

9. A formulation as in claim 1 wherein the ingredient percentages are:

| | |
|---|---|
| white emulsion | 76.25% by wt |
| bemotrizinol | 10% by wt |
| octyl salicylate | 5% by wt |
| octyl methoxycinnamate | 5% by wt |
| titanium dioxide | 0.75% by wt |

further including 3% by wt CoQ10.

10. A formulation as in claim 1 wherein the ingredient percentages are:

| white emulsion | 70.25% by wt |
|---|---|
| bemotrizinol | 10% by wt |
| octyl salicylate | 6% by wt |
| octyl methoxycinnamate | 6% by wt |
| titanium dioxide | 0.75% |

further including 3% by wt CoQ10 and 4% by wt vanillin.
